(19) United States
(12) Patent Application Publication
(10) Pub. No.: US 2024/0157089 A1
(43) Pub. Date: May 16, 2024

(54) DUAL BALLOON CATHETER INFUSION SYSTEM

(71) Applicant: Merit Medical Systems, Inc., South Jordan, UT (US)

(72) Inventors: Jason Wiersdorf, West Jordan, UT (US); Dillon Despain, Bluffdale, UT (US)

(21) Appl. No.: 18/506,458

(22) Filed: Nov. 10, 2023

Related U.S. Application Data
(60) Provisional application No. 63/383,330, filed on Nov. 11, 2022.

(51) Int. Cl. A61M 25/00; A61M 25/10

(52) U.S. Cl. CPC A61M 25/0075; A61M 25/10; A61M 2025/105; A61M 2025/1052

(57) ABSTRACT

Systems and methods used to treat a vessel are disclosed. The systems include an occlusion catheter device having a balloon disposed at a distal end and a connector coupled to a proximal end of the catheter. A handle having a slide actuator is coupled to the connector. A seal wire is displaceable by the slide actuator to allow deflation of the balloon through a vent port. The system includes a catheter infusion device that is coaxially disposed over the occlusion catheter device and having a balloon disposed at a distal end and a connector coupled to a proximal end. A backflow prevention member is coupled to the connector and is configured to seal around the occlusion catheter device.

### RELATED APPLICATIONS

This application claims priority to U.S. Provisional Application No. 63/383,330, filed on Nov. 11, 2022 and titled, “Dual Balloon Catheter Infusion System,” which is hereby incorporated by reference in its entirety.

### TECHNICAL FIELD

The present disclosure relates generally to devices to treat organs intravascularly. More specifically, the present disclosure relates to a micro balloon catheter device used to occlude a patient's blood vessel.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a catheter infusion system.

FIG. 2 is a perspective view of an embodiment of a catheter occlusion device of the catheter infusion system of FIG. 1 in an inflated state.

FIG. 3 is a cross-sectional side view of a proximal portion of the catheter occlusion device of FIG. 2.

FIG. 4A is cross-sectional side view of a distal portion of the catheter occlusion device of FIG. 2.

FIG. 4B is a cross-sectional view of the distal portion of the catheter occlusion device of FIG. 3 through section line 3A-3A.

FIG. 5A is a side view of the catheter occlusion device of FIG. 2 inserted into a patient's blood vessel in a ready state.

FIG. 5B is a side view of a distal portion of the catheter occlusion device of FIG. 5A.

FIG. 6A is a side view of the catheter occlusion device of FIG. 2 inserted into a patient's catheter vessel in an inflated state.

FIG. 6B is a side view of a distal portion of the catheter occlusion device of FIG. 6A.

FIG. 7A is a side view of the catheter occlusion device of FIG. 2 inserted into a patient's blood vessel in a deflated state.

FIG. 7B is a side view of a distal portion of the catheter occlusion device of FIG. 7A.

FIG. 8 is a perspective view of an embodiment of a catheter infusion device.

FIG. 9A is a side cross-sectional view of a proximal portion of the catheter infusion device of FIG. 8.

FIG. 9B is a side cross-sectional view of a distal portion of the catheter infusion device of FIG. 8.

FIG. 10 is a transverse cross-sectional view of the distal portion of the catheter infusion system of FIG. 1 through section 10-10.

FIG. 11A is a side view of the catheter infusion system of FIG. 1 inserted into a patient's blood vessel in a ready state.

FIG. 11B is a side view of the catheter infusion system of FIG. 1 inserted into a patient's blood vessel in a fluid infusion state.

FIG. 11C is a side view of the catheter infusion system of FIG. 1 inserted into a patient's blood vessel in a deflated state.

FIG. 12A illustrates another embodiment of the catheter occlusion device in an assembled state.

FIG. 12B illustrates the catheter occlusion device of FIG. 12A in a disassembled state.

### DETAILED DESCRIPTION

In certain instances, a diseased patient organ can be treated intravascularly using targeted delivery of a treatment substance. For example, an organ having a tumor or cancerous growth may be treated by intravascular targeted delivery of a chemotherapeutic drug. In another example, a blood vessel within an organ can be permanently occluded to prevent blood flow into a tumor by targeted delivery of a thrombogenic agent. The intravascular treatment can utilize a dual balloon catheter infusion system that includes a micro balloon catheter to occlude the blood vessel distal of the treatment site and prevent flow of the treatment substance from the treatment site and a fluid infusion catheter having a balloon disposed over the micro balloon catheter.

Embodiments herein describe infusion catheter systems and methods to assist in targeted intravascular delivery of a treatment substance. The systems can be percutaneously inserted into a blood vessel of the organ or body area to be treated. In some embodiments within the scope of this disclosure, the infusion catheter systems include a catheter occlusion device that includes an elongate catheter or tube coupled to a connector having an inflation port. An inflation or expandable member (e.g., balloon) is disposed adjacent a distal end of the catheter. The balloon is in fluid communication with the inflation port through the catheter wherein the balloon can be inflated or expanded with a fluid. A vent port is coupled to the distal end of the catheter. A bore extends through the vent port and is in fluid communication with the balloon. The bore has a proximal portion and a distal portion. A diameter of the proximal portion is larger than a diameter of the distal portion. The bore has a distal opening.

A handle or actuator is couplable to the connector. The handle includes a slide actuator disposed within a housing. The slide actuator is displaceable between a distal position and a proximal position. A seal wire is coupled to the slide actuator and is displaced by the slide actuator. When the slide actuator is in the distal position, a distal end of the seal wire is sealingly disposed within the distal portion of the bore of the vent port to prevent fluid from flowing from the balloon and out the opening. This configuration allows the balloon to be inflated or expanded by the fluid delivered from the inflation port. When the slide actuator is in the proximal position, the distal end of the seal wire is disposed within the proximal portion of the bore of the vent port. This configuration allows fluid from the balloon to flow through the bore and out the opening resulting in self-deflation of the balloon within a short period of time.

In some embodiments within the scope of this disclosure the infusion catheter systems include an infusion catheter device that is coaxially disposable over the catheter occlusion device. The infusion catheter device includes an elongate catheter or tube coupled to a connector having an inflation port. An inflation or expandable member (e.g., balloon) is disposed adjacent a distal end of the catheter. The balloon is in fluid communication with the inflation port through an annular space of the catheter wherein the balloon can be inflated or expanded with a fluid. A backflow valve device is couplable to the connector and configured to prevent backflow of fluid, including blood, from the infusion catheter device.

In use, in embodiments within the scope of this disclosure, the catheter occlusion device and the infusion catheter device are percutaneously inserted into the blood vessel of the organ to be treated such that the distal end of the vessel occlusion device is positioned adjacent a desired treatment site. When inserted, the catheter occlusion device is in a ready state where the balloon is deflated, the slide actuator is in the distal position, and the distal end of the seal wire is sealingly disposed within the distal portion of the bore of the vent port. The balloon may be inflated or expanded to occlude the vessel distal of a treatment site by injection of a fluid from a fluid delivery device coupled to the inflation port. The fluid can flow through the inflation port, through an annular space within the catheter defined by the seal wire and the catheter wall, through a side port of the catheter, and into the balloon. The seal of the seal wire within the distal portion of the bore prevents fluid from flowing out the distal opening of the vent port.

The balloon of the infusion catheter device may be inflated or expanded to occlude the vessel proximal of the treatment site by injection of a fluid from a fluid delivery device coupled to the inflation port. The fluid can flow through the inflation port, through the annular space within the catheter, and into the balloon. A treatment medicament can be injected into the treatment site from a fluid delivery device through an infusion port of the backflow valve device and the catheter of the infusion catheter device. Treatment medicaments including drugs, solutions, and embolic particles are all within the scope of this disclosure.

The balloon of the catheter occlusion device may be deflated by displacement of the slide actuator to the proximal position resulting in proximal displacement of the seal wire from within the distal portion to the proximal portion of the bore of the vent port. This configuration allows fluid to flow from the balloon, through the side port, distally through the annular space, through the proximal portion around the seal wire, through the distal portion, and out the distal opening resulting in self-deflation of the balloon within a short time period of one to five seconds.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

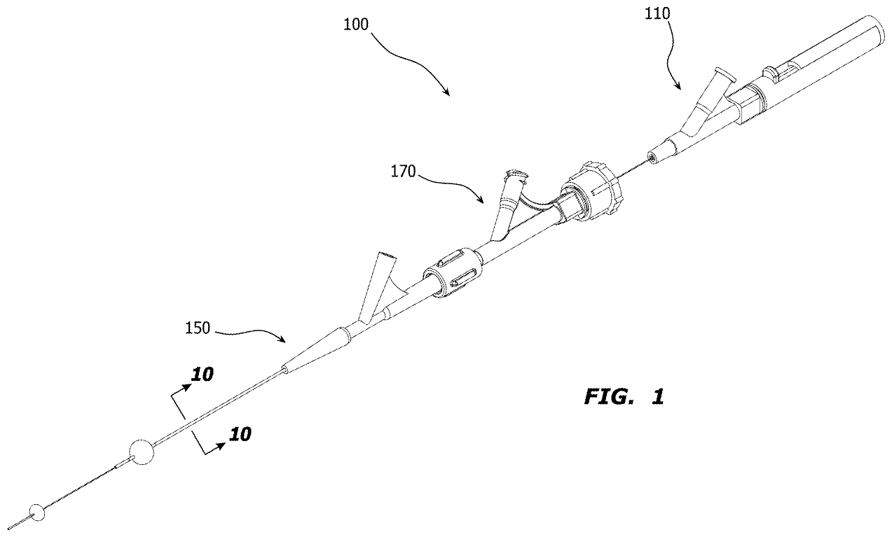

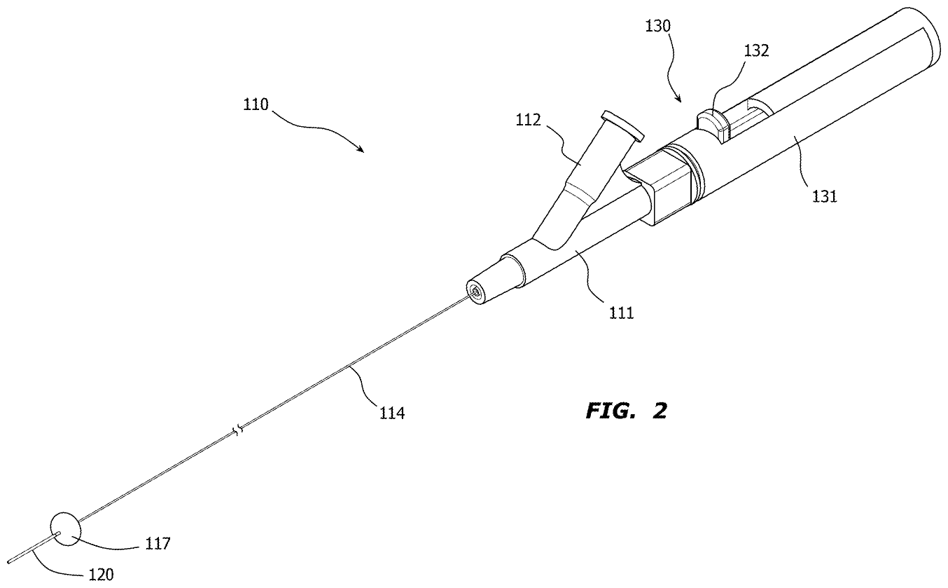

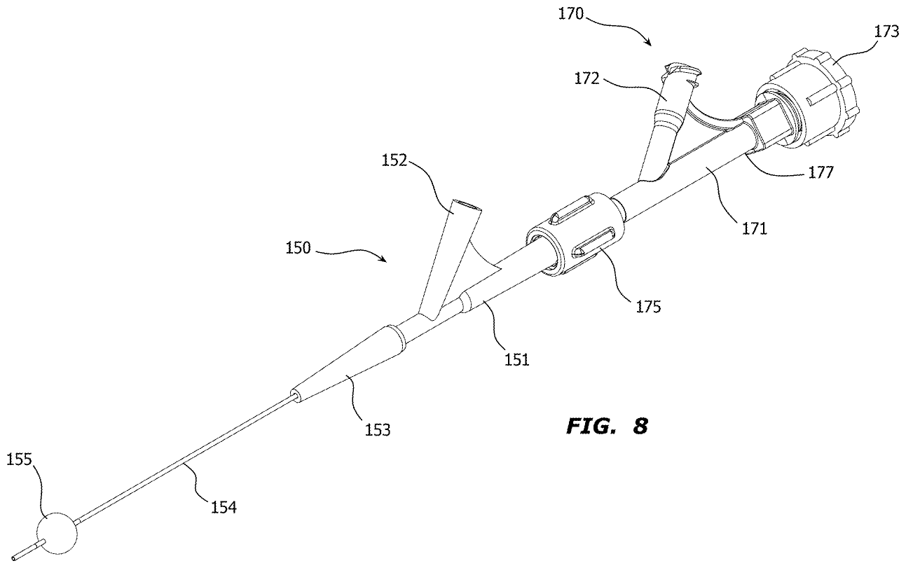

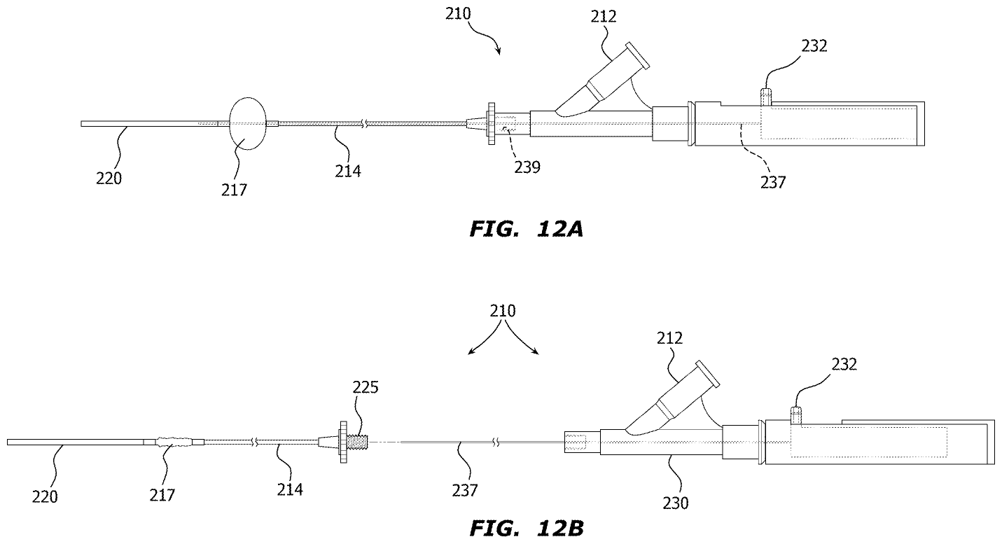

FIGS. 1-11C illustrate an embodiment of a catheter infusion system with FIGS. 2-7B illustrating an embodiment of a catheter occlusion device and FIGS. 8-11C illustrating an embodiment of an infusion catheter device. FIGS. 12A and 12B illustrate another embodiment of a catheter occlusion device. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

As illustrated in FIG. 1, a catheter infusion system 100 includes three broad groups of components; each group may have numerous subcomponents and parts. The three broad component groups are: a first balloon catheter or catheter occlusion device 110, a second balloon catheter or infusion catheter device 150, and a backflow valve device 170. In the illustrated embodiment of FIG. 1, the infusion catheter device 150 and the backflow valve device 170 are coaxially disposed over the catheter occlusion device 110. The backflow valve device 170 is coupled to the infusion catheter device 150 and disposed between the catheter occlusion device 110 and the infusion catheter device 150.

As depicted in FIGS. 2-4A, the catheter occlusion device 110 includes a connector 111, an elongate tube or catheter 114, an expandable or inflatable vessel occlusion member 117, and a vent port or fluid evacuation port 120. The connector 111 is coupled to a proximal portion of the catheter 114 and may have a “Y” shape. An inflation port 112 is in fluid communication with a lumen 115 of the catheter 114 wherein a fluid can be injected from a fluid delivery device (e.g., syringe) coupled to the inflation port 112 to inflate the inflatable or expandable vessel occlusion member 117. In certain embodiments, the connector 111 is fixedly coupled to the catheter 114. In other embodiments, the connector 111 may be selectively coupled to the catheter 114, wherein the connector 111 may be decoupled from the catheter 114 to allow placement of a catheter, such as an infusion catheter, over the catheter 114 to accomplish a desired medical procedure.

The catheter 114 extends distally from the connector 111. The catheter 114 may be formed of a shape memory metal alloy, such as nitinol, or any other suitable material that allows the catheter 114 to be maneuvered through a tortuous vessel path. An outer diameter of the catheter 114 may range from about 0.25 millimeters to about 0.5 millimeters, and may be about 0.27 millimeters. The lumen 115 extends through the catheter 114 in alignment with a longitudinal axis of the catheter 114. A diameter of the lumen 115 may range from about 0.18 millimeters to about 0.35 millimeters, and may be about 0.18 millimeters.

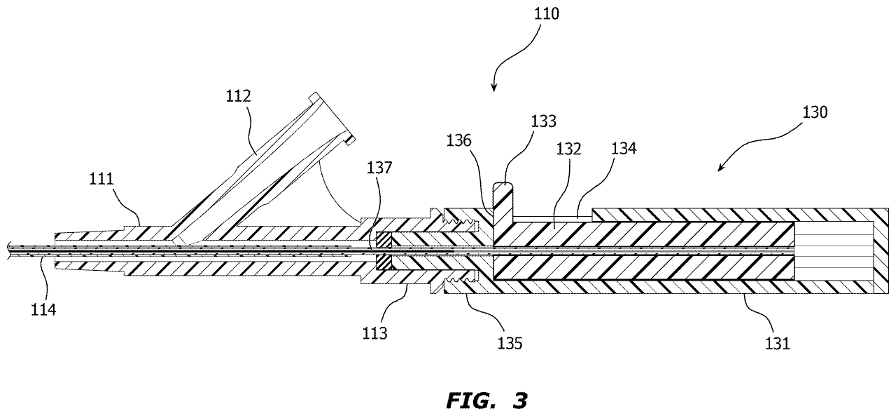

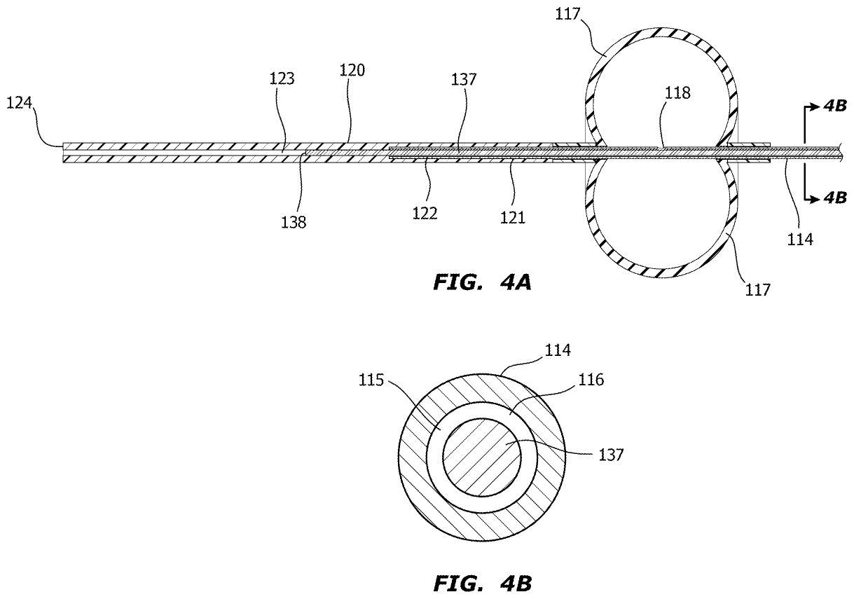

As shown in FIG. 4A, the expandable or inflatable vessel occlusion member 117 is coupled to the catheter 114 adjacent a distal end using any suitable technique, such as welding, bonding, gluing, etc. The occlusion member 117 may be a balloon formed of any suitable elastomeric material configured to elongate and withstand rupturing at a fluid pressure ranging from about 5 pounds per square inch to about 24 pounds per square inch. For example, the occlusion member 117 may be formed of silicone, polyisoprene or an aromatic polyether based urethane and a styrene blocked copolymer. The occlusion member 117 is configured to have a deflated or non-expanded state wherein a diameter of the occlusion member 117 is substantially equivalent to the outer diameter of the catheter 114 in an inflated or expanded state, as shown in FIG. 3, wherein the diameter of the occlusion member 117 may range from about 0.5 millimeters to about 5 millimeters dependent upon the material, wall thickness, and fluid pressure applied to the occlusion member 117. When in the inflated state, the occlusion member 117 can occlude or block blood flow within a blood vessel as the occlusion member 117 engages with the wall of the blood vessel. In another embodiment, when in the inflated state, the occlusion member 117 may anchor the catheter 114 in place relative to the blood vessel as an infusion catheter is placed over the catheter 114. One or more additional catheters or devices may be advance along the catheter 114 or other portions of the assembly when the catheter 114 is so anchored.

The catheter 114 includes a side port 118 disposed through the wall of the catheter 114 and positioned within the occlusion member 117 to provide fluid communication between the lumen 115 and the occlusion member 117. When the occlusion member 117 is inflated or expanded, fluid flows through the inflation port 112, through the lumen 115, through the side port 118, and into the occlusion member 117.

The vent port 120 is coupled to a distal end of the catheter 114 and disposed distally of the occlusion member 117. The vent port 120 may be formed of any suitable polymeric material, such as pebax or nylon. In some embodiments, the vent port 120 may be flexible to permit passage of the catheter 114 through a tortuous vessel. In another embodiment, the vent port 120 and the catheter 114 may include a steering wire, such as a flat wire, extending from the vent port 120, along the catheter 114, and to a steering mechanism (e.g., wire tensioning mechanism) disposed at a proximal end of the catheter 114. The steering wire and the steering mechanism may be utilized to deflect the vent port 120 into an arcuate shape to allow steering of the catheter 114 through the tortuous vessel. Coupling of the vent port 120 to the catheter 114 can be accomplished using any suitable manufacturing technique, such as welding, bonding, gluing, overmolding, etc. An outer diameter of the vent port 120 may be substantially equivalent to the outer diameter of the catheter 114.

A bore 121 extends longitudinally through the vent port 120. The bore 121 is in axial alignment and fluid communication with the lumen 115. The bore 121 includes a first or proximal portion 122 having a diameter ranging from about 0.18 millimeters to about 0.35 millimeters and a second or distal portion 123 which may have a smaller diameter. The bore 121 includes an opening 124 disposed at a distal end of the vent port 120. When the occlusion member 117 is deflated, the fluid within the occlusion member 117 can flow through the side port 118, through the lumen 115, through the proximal portion 122, through the distal portion 123, and through the opening 124 into the environment surrounding the vent port 120, such as the blood within the blood vessel.

As illustrated in FIGS. 2-4B, the handle 130 includes a housing 131, a slider or slide member or actuator 132 slidingly disposed within the housing 131, and a seal wire 137. The slider 132 includes a tab 133 extending radially outward from a longitudinal axis of the slider 132. The tab 133 is disposed within a slot 134 of the housing 131. The slot 134 allows the slider 132 to be displaced between a distal position, as shown in FIG. 2, and a proximal position when a user engages the tab 133 with one or more fingers. The slot 134 includes a positive distal stop 136 to stop distal movement of the slider 132 relative to the housing 131. A distal end 135 of the housing 131 is couplable to a proximal end 113 of the connector 111. The distal end 135 may include internal threads to engage with external threads of the proximal end 113. In other embodiments, the housing 131 may be coupled to the connector 111 using any suitable technique, such as a snap fit, a friction fit, etc.

The seal wire 137 is operably coupled to the slider 132 and extends distally from the slider 132 through the connector 111. The seal wire 137 may be formed of any suitable material, such as stainless steel, shape memory metal alloy (e.g., nitinol), etc. A diameter of the seal wire 137 may range between about 0.13 millimeters and about 0.3 millimeters. When the slider 132 is displaced a distance proximally from the distal position, the seal wire 137 is displaced proximally an equal distance, and when the slider 132 is displaced a distance distally from the proximal position, the seal wire 137 is displaced distally an equal distance. The seal wire 137 is coaxially disposed within the lumen 115 of the catheter 114 to define an annular space 116 between the seal wire 137 and the wall of the catheter 114, as shown in FIG. 4B. A cross-sectional area of the annular space 116, may range from about 0.004 millimeters 2 to about 0.008 millimeters 2 which is smaller than a cross-sectional area of distal portion 123 of the bore 121. This configuration provides for less fluid flow resistance through the distal portion 123 than through the annular space 116 resulting in fluid flowing easier from the occlusion member 117 through the vent port 120 than from the occlusion member 117 through the annular space 116 during deflation of the occlusion member 117. The inflation port 112 and the occlusion member 117 are in fluid communication with the annular space 116 such that when the occlusion member 117 is inflated or expanded, fluid can flow through the inflation port 112, through the annular space 116, through the side port 118, and into the occlusion member 117.

As depicted in FIG. 4A, the seal wire 137 extends into the bore 121 of the vent port 120. When the slider 132 is in the distal position, a distal portion 138 of the seal wire 137 is disposed within the second portion 123 and seals against an inner surface of the second portion 123 to prevent fluid from flowing through the bore 121 allowing the occlusion member 117 to be inflated and to maintain the inflated state. The positive distal stop 136 can prevent distal displacement of the seal wire 137 that results in the distal portion 138 extending distally from the vent port 120. When the slider 132 is in the proximal position, the distal portion 138 is disposed within the first portion 122 allowing fluid to flow from the occlusion member 117 as it deflates, through the side port 118, through the annular space 116, through the first portion 122 around the seal wire 137, through the second portion 123, and out the opening 124 into the environment surrounding the vent port 120.

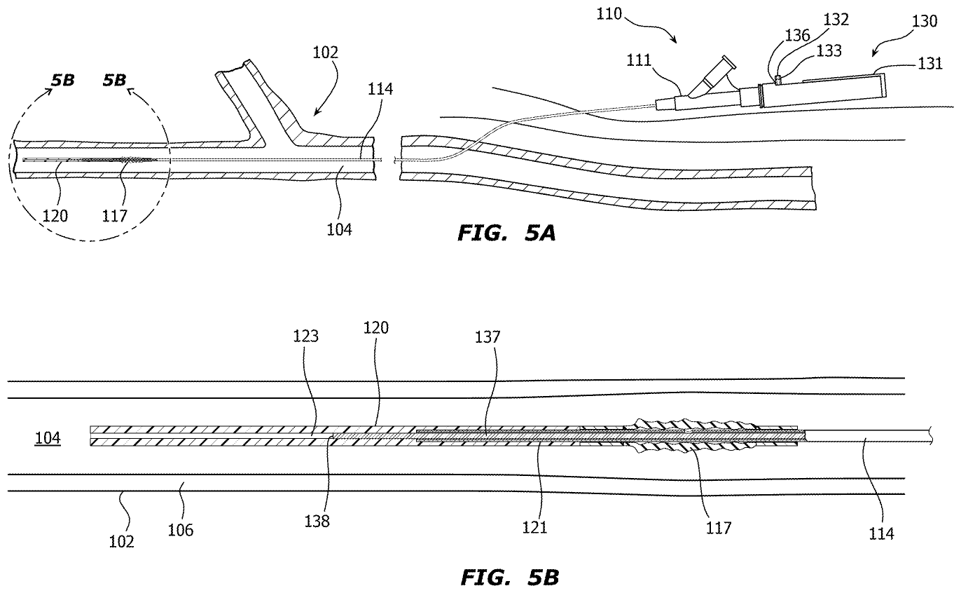

FIGS. 5A-7B illustrate the catheter occlusion device 110 in use. As illustrated in FIGS. 5A and 5B, the catheter occlusion device 110 is in a ready state and percutaneously inserted through a patient's skin into a lumen 104 of a patient's blood vessel 102. For example, the catheter occlusion device 110 may be inserted into a blood vessel of a patient's liver, pancreas, neck, or leg. Other blood vessels are contemplated within the scope of this disclosure. The flexibility of the vent port 120 may allow the catheter 114 to be guided through tortuous vessels to a desired treatment location. In another embodiment, the catheter occlusion device 110 may include a steering wire and a steering mechanism, as previously described, to help guide or steer the catheter 114 through the tortuous vessels.

The handle 130 is coupled to the connector 111 of the catheter occlusion device 110 with the slider 132 in the distal position wherein the tab 133 is disposed against the positive distal stop 136 of the housing 131 to position the distal portion 138 of the seal wire 137 within the distal portion 123 of the bore 121 of vent port 120. The vessel occlusion member 117 is in a deflated or non-expanded state wherein the outer diameter is substantially equivalent to the outer diameter of the catheter 114 and the vent port 120.

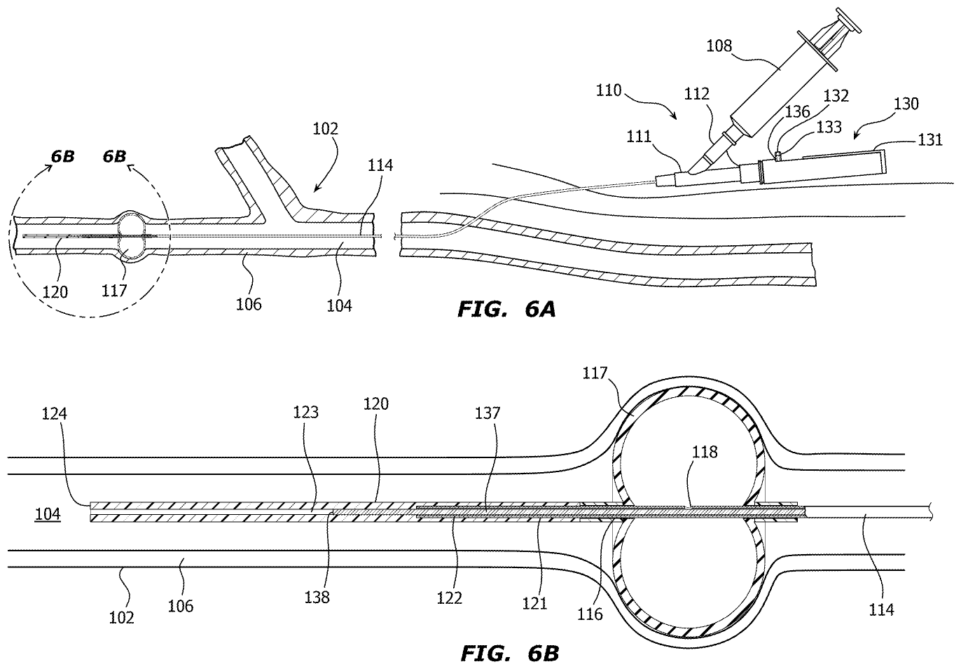

FIGS. 6A and 6B illustrate the catheter occlusion device 110 in a vessel occluding state wherein the occlusion member 117 is inflated against a wall 106 of the vessel 102 to occlude or block blood flow through the lumen 104 of the vessel 102. The handle 130 is coupled to the connector 111 of the catheter occlusion device 110 with the slider 132 in the distal position wherein the tab 133 is disposed against the positive distal stop 136 of the housing 131 to position the distal portion 138 of the seal wire 137 within the distal portion 123 of the bore 121 of the vent port 120. When disposed within the distal portion 123, the seal wire 137 seals the distal portion 123 to prevent or restrict fluid from flowing through the distal portion 123 when the occlusion member 117 is inflated or expanded with a fluid. The positive distal stop 136 may also prevent the seal wire 137 from extending distally from the opening 124 of the vent port 120.

A fluid delivery device 108 (e.g., syringe) is coupled to the inflation port 112 of the connector 111. The fluid delivery device 108 may be at least partially filled with any suitable fluid configured to inflate or expand the occlusion member 117. For example, the fluid may be saline, contrast media, a saline and contrast media mixture, etc. The fluid is injected into the catheter occlusion device 110 by the fluid delivery device 108 wherein the fluid flows through the inflation port 112, the annular space 116, the side port 118, and into the occlusion member 117 resulting in inflation or expansion of the occlusion member 117. The occlusion member 117 may be inflated or expanded at a fluid pressure ranging from about 5 pounds per square inch to about 19 pounds per square inch.

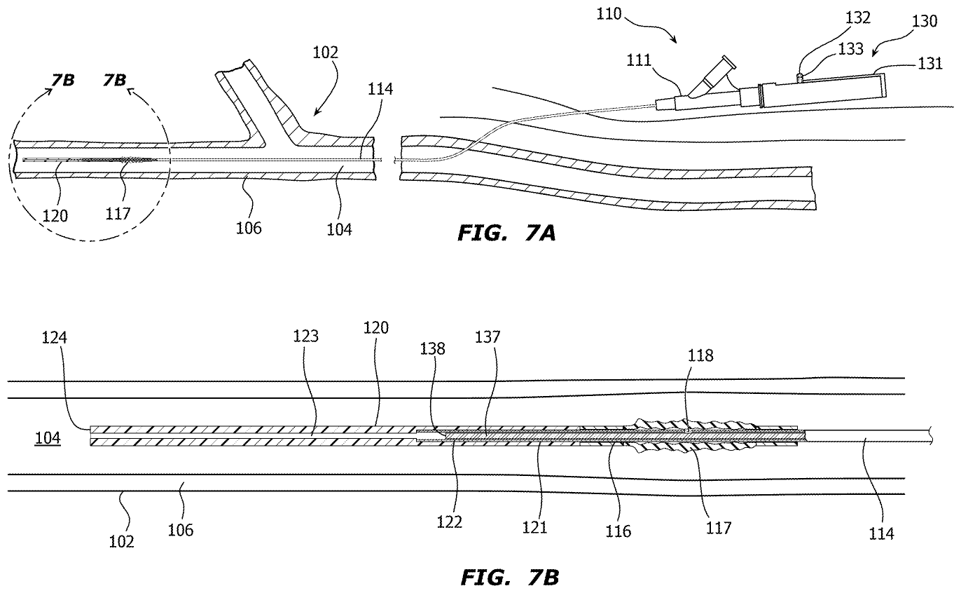

FIGS. 7A and 7B illustrate the catheter occlusion device 110 in a deflated state where the occlusion member 117 is deflated to allow blood flow through the lumen 104 of the vessel 102. As illustrated, the handle 130 is coupled to the connector 111 of the catheter occlusion device 110. The slider 132 is displaced to the proximal position within the housing 131. The distal portion 138 of the seal wire 137 is displaced into the proximal portion 122 of the bore 121 of the vent port 120. When the distal portion 138 is disposed within the proximal portion 122, the distal portion 123 is open to allow fluid to flow from the occlusion member 117, through the side port 118, through the annular space 116, through the proximal portion 122 around the seal wire 137, through the distal portion 123, through the opening 124, and into the lumen 104 of the blood vessel 102. The fluid flow can be pressurized by the elastomeric contraction of the occlusion member 117 and may flow out the opening 124 due to less distal flow resistance than proximal flow resistance. The occlusion member 117 may self-deflate over a time ranging from about one second to about five seconds.

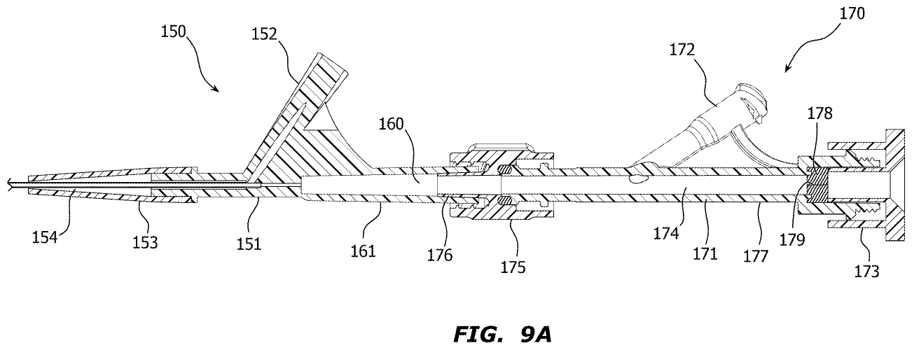

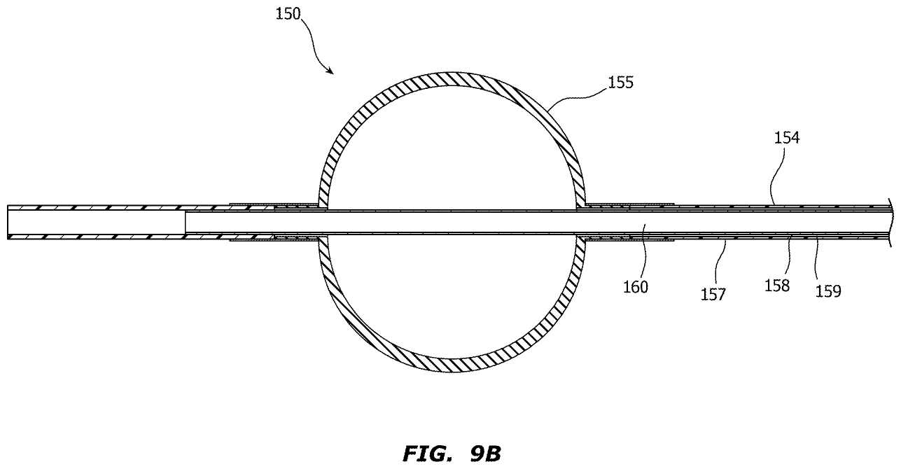

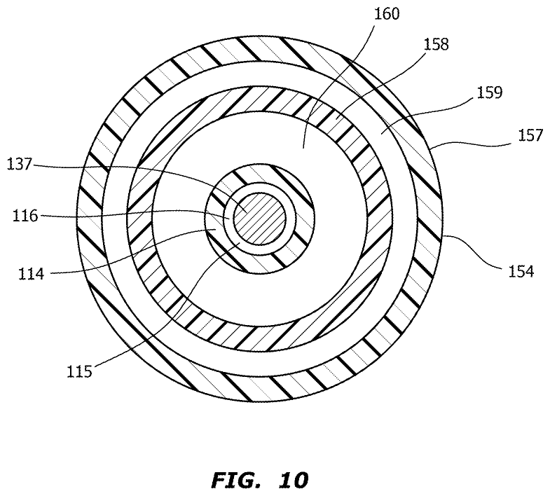

As illustrated in FIGS. 8, 9A, 9B, and 10, the infusion catheter device 150 includes a connector 151 coupled to an elongate catheter 154. The connector includes an inflation port 152 and a primary port 161. As depicted, the connector 151 has a “Y” shape. In other embodiments, the connector 151 may have any suitable shape, such as a “T” shape. A strain relief member 153 is coupled to a distal end of the connector 151 and coaxially disposed over the catheter 154 to prevent kinking of the catheter 154 at the distal end of the connector 151. As shown in FIGS. 9B and 10, the catheter 154 includes an outer tube 157 and an inner tube 158 coaxially disposed within the outer tube 157. The outer tube 157 can be coupled to the connector 151. The outer tube 157 may have an outer diameter ranging from about 1 millimeters to about 3 millimeters. An outer diameter of the inner tube 158 is smaller than an inner diameter of the outer tube 157 wherein an annular space 159 is defined between the outer tube 157 and the inner tube 158 as shown in FIG. 10. The outer diameter of the inner tube 158 may range from about 0.5 millimeters to about 2.5 millimeters. The annular space 159 is in fluid communication with the inflation port 152. A lumen 160 of the inner tube 158 is axially aligned with the primary port 161. The lumen 160 extends along a length of the inner tube 158.

An expandable or inflatable vessel occlusion member 155 (shown in the expanded or inflated state in FIGS. 8 and 9B) is coupled to the catheter 114 adjacent a distal end 135 of the catheter 154 using any suitable technique, such as welding, bonding, gluing, etc. The occlusion member 155 may be a balloon formed of any suitable elastomeric material configured to elongate and withstand rupturing at a fluid pressure ranging from about 5 pounds per square inch to about 24 pounds per square inch. For example, the occlusion member 155 may be formed of silicone, polyisoprene or an aromatic polyether based urethane and a styrene blocked copolymer. The occlusion member 155 is configured to have a deflated or non-expanded state wherein a diameter of the occlusion member 155 is substantially equivalent to the outer diameter of the outer tube 157 and the inflated or expanded state wherein the diameter of the occlusion member 155 may range from about 3 millimeters to about 10 millimeters dependent upon the fluid pressure applied to the interior of the occlusion member 155. When in the inflated state, the occlusion member 155 can occlude or block blood flow within a blood vessel as the occlusion member 155 engages with the wall of the blood vessel.

As illustrated in FIGS. 8 and 9A, the backflow valve device 170 is coupled to the primary port 161. In the depicted embodiment, the backflow valve device 170 is a Touhy-Borst type adapter. In other embodiments, the backflow valve device 170 may be any suitable type of device configured to prevent backflow of fluid when a catheter or wire is disposed through the backflow valve device 170. Other designs and configurations of valves are within the scope of this disclosure. The backflow valve device 170 includes a body 171 having a “Y” shape. In other embodiments, the body 171 may have any suitable shape, such as a “T” shape. The body 171 includes an infusion port 172 and a primary port 177 having a lumen 174. The infusion port 172 is in fluid communication with the lumen 160 wherein a medicament can be injected from a fluid delivery device, through the infusion port 172, through the lumen 174, through the lumen 160, through the catheter 154, and into the lumen of the vessel at the treatment site. A valve member 178 having a bore 179 therethrough is disposed within the primary port 177. A valve compression cap 173 is threadingly coupled to the primary port 177 proximal to the valve member 178. The valve compression cap 173 can be rotated in a first direction (e.g., clockwise) to longitudinally compress the valve member 178 resulting in narrowing of the bore 179. In some embodiments, narrowing of the bore 179 may result in sealing of the valve member 178 to prevent backflow of fluid through the backflow valve device 170. The backflow valve device 170 may include a male Luer fitting 176 and a male Luer nut 175 to facilitate coupling of the backflow valve device 170 to the primary port 161.

FIG. 10 illustrates a cross-section of the catheter infusion system 100 at section line 10-10 of FIG. 7. As shown, the annular space 159 is defined as the space between the outer tube 157 and the inner tube 158 of the catheter 154. The catheter 114 is coaxially disposed within the lumen 160 and the seal wire 137 is disposed within the catheter 114. This configuration provides for fluid to inflate or expand the occlusion member 155, fluid to inflate or expand the vessel occlusion member 117, and a medicament fluid to flow through the lumen 160 without any type of intermixing of the fluids. In other words, the fluids are completely isolated from each other as they flow through the catheter infusion system 100.

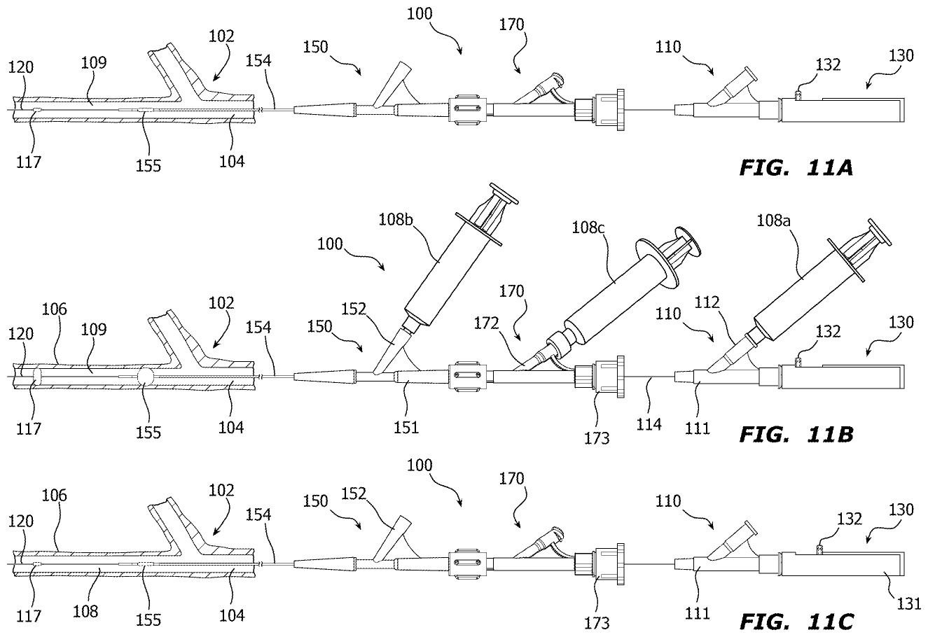

FIGS. 11A-11C illustrate the catheter infusion system 100 in use. As illustrated in FIG. 11A, the catheter infusion system 100 is in a ready state and percutaneously inserted through a patient's skin into a lumen 104 of a patient's blood vessel 102. For example, the catheter infusion system 100 may be inserted into a blood vessel of a patient's liver, pancreas, neck, or leg. Other blood vessels are contemplated within the scope of this disclosure. The infusion catheter device 150 and the backflow valve device 170 are coaxially disposed over the catheter 114 of the catheter occluding device 110. The catheter occlusion device 110 is inserted into the blood vessel 102 until the first vessel occlusion member 117 is positioned at a desired location relative to a treatment site. The catheter infusion device 150 can be axially displaced relative to catheter occlusion device 110 to position the second vessel occlusion member 155 at a desired location relative to the treatment site and the first vessel occlusion member 117. A distance between the first and second vessel occlusion members 117, 155 can be adjusted to define a treatment segment 109 of the vessel lumen 104. In some embodiments, a length of the treatment segment 109 can range between 0.5 centimeters and 20 centimeters. The first and second occlusion members 117, 155 are in a deflated state. The slider 132 of the handle 130 is in a distal position wherein a seal wire seals the vent port 120, as previously described.

FIG. 11B illustrates the catheter infusion system 100 in a fluid infusion state wherein the occlusion members 117, 155 are in inflated states against a wall 106 of the vessel 102 to occlude or block blood flow through the lumen 104. The slider 132 of the handle 130 is in a distal position wherein a seal wire seals the vent port 120, as previously described. The valve compression cap 173 is rotated in the first direction to longitudinally compress a valve member within the backflow valve device 170. When the valve member is compressed, a diameter of a bore of the valve member is reduced. In some embodiments, the valve member seals around the catheter 114 when compressed to prevent of backflow of fluid (e.g., medicament, blood) and to prevent axial and rotational movement of the catheter occlusion device 110 relative to the catheter infusion device 150.

A fluid delivery device 108a (e.g., syringe) is coupled to the inflation port 112 of the connector 111. The fluid delivery device 108a may be at least partially filled with any suitable fluid configured to inflate or expand the first occlusion member 117. The fluid is injected into the catheter occlusion device 110 by the fluid delivery device 108a wherein the fluid flows through the inflation port 112, into an annular space defined by the catheter 114 and the seal wire, and into the first occlusion member 117 resulting in inflation or expansion of the first occlusion member 117.

A fluid delivery device 108b (e.g., syringe) is coupled to the inflation port 152 of the connector 151. The fluid delivery device 108b may be at least partially filled with any suitable fluid configured to inflate or expand the second occlusion member 155. The fluid is injected into the catheter infusion device 150 by the fluid delivery device 108b wherein the fluid flows through the inflation port 152, into an annular space of the catheter 154, and into the second occlusion member 155 resulting in inflation or expansion of the first occlusion member 155.

A fluid delivery device 108c (e.g., syringe) is coupled to the infusion port 172 of the body 171. The fluid delivery device 108c may be at least partially filled with any suitable medicament configured to occlude or treat the treatment site. For example, the medicament may include a thrombogenic agent or particles, a chemotherapeutic agent, etc. The medicament is injected into the catheter infusion device 150 by the fluid delivery device 108c wherein the medicament flows through the infusion port 172, the connector 151, and a lumen of the catheter 154, and into the treatment segment 109. In some embodiments, one or more branch vessels may extend from the defined or isolated treatment segment 109, which may facilitate treatment of the vessel 102 along the treatment segment 109 as well as branches downstream from the vessel 102 that intersect with the treatment segment. Embodiments within the scope of this disclosure wherein treatment is configured to be delivered only to an isolated treatment segment 109 (e.g. with no branch vessels and occlusion members on either side of the treatment segment) and embodiments wherein treatment is applied to a treatment segment 109 and portions of the vasculature downstream from the treatment segment are contemplated.

FIG. 11C illustrates the catheter infusion system 100 in a deflated state where the occlusion members 117, 155 are deflated to allow blood flow through the lumen 104 of the vessel 102. As illustrated, the handle 130 is coupled to the connector 111. The slider 132 is displaced to the proximal position within the housing 131. A distal end of the seal wire is displaced proximally to allow fluid to flow from the first occlusion member 117, through the vent port 120, and into the lumen 104 of the blood vessel 102, as previously described. The first occlusion member 117 may self-deflate over a time ranging from about one second to about five seconds. The second occlusion member 155 is deflated by flow of the fluid within the second occlusion member 155 through the annular space of the catheter 154 and the inflation port 152. When the occlusion members 117, 155 are in the deflated state, the catheter infusion system 100 can be removed from the vessel 102.

FIGS. 12A and 12B depict an embodiment of a catheter occlusion device 210 that resembles the catheter occlusion device 110 described above in certain respects. Accordingly, like features are designated with like reference numerals, with the leading digit incremented to “2.” For example, the embodiment depicted in FIGS. 12A and 12B includes a handle 230 that may, in some respects, resemble the handle 130 of FIG. 2. Relevant disclosure set forth above regarding similarly identified features thus may not be repeated hereafter. Moreover, specific features of the catheter occlusion device 110 and related components shown in FIGS. 2-7B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the catheter occlusion device 210 and related components depicted in FIGS. 12A and 12B. Any suitable combination of the features, and variations of the same, described with respect to the catheter occlusion device 110 and related components illustrated in FIGS. 2-7B can be employed with the catheter occlusion device 210 and related components of FIGS. 12A and 12B, and vice versa. This pattern of disclosure applies equally to further embodiments depicted in subsequent figures and described hereafter, wherein the leading digits may be further incremented.

As illustrated in FIGS. 12A and 12B, the catheter occlusion device 210 includes a catheter 214 releasably coupled to a handle 230. An expandable or inflatable occlusion member 217 is coupled to the catheter 214 and disposed proximal to a vent port 220 disposed at a proximal end of the catheter 214. The handle 230 includes an inflation port 212 in fluid communication with the occlusion member 217. A slider or slide member 232 is slidingly disposed within a proximal portion of the handle 230. The slider 232 is configured to axially displace a seal wire 237 coupled to the slider 232 between a distal position and a proximal position, as previously described related to the catheter occlusion device 110.

A catheter fitting 225 is coupled to a proximal end of the catheter 214. The catheter fitting 225 is configured to selectively couple with a handle fitting 239 of the handle 230 wherein a fluid-tight seal is provided between the catheter fitting 225 and the handle fitting 239. In the depicted embodiment, the catheter fitting 225 includes external threads that are threadingly engageable with internal threads of the handle fitting 239. In other embodiments, the catheter fitting 225 and the handle fitting 239 may include any suitable coupling features to enable selective coupling and de-coupling of the catheter 214 to the handle 230. For example, the coupling features may include male and female tapered fittings, snap fit fittings, and bayonet fittings. Other types of coupling features are contemplated within the scope of this disclosure.

The selective coupling of the catheter 214 to the handle 230 allows a user to remove the handle 230 from the catheter 214 and dispose a catheter (e.g., infusion catheter) over the catheter 214 from the proximal end of the catheter 214. For example, in use, the catheter 214 may be inserted into a patient's vessel with the occlusion member positioned at a desired treatment site. The handle 230 can be de-coupled from the catheter 214 and the infusion catheter coaxially disposed over the catheter 214 from the proximal end toward the distal end of the catheter 214. The handle 230 can be re-coupled to the catheter 214, the occlusion member 217 expanded or inflated with a fluid injected through the inflation port 212, and a treatment medicament infused into the patient's vessel proximal to the occlusion member 217 through the infusion catheter.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of intravascular treatment may include one or more of the following steps: disposing a catheter occlusion device coaxially over a catheter infusion device; disposing an actuator of the catheter occlusion device in a first position, wherein a seal member seals a bore of an occlusion member evacuation port of the catheter occlusion device; inflating a first expandable occlusion member of the catheter occlusion device with a fluid to occlude a vessel; inflating a second expandable occlusion member of the catheter infusion device with a fluid to occlude the vessel; moving the actuator from the first position to a second position, wherein the seal member is displaced to open the bore; and deflating the first expandable occlusion member, wherein the fluid flows from the first expandable occlusion member into the bore and out a distal end of the occlusion member evacuation port. Other steps are also contemplated.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use. As specifically applied to a vessel occluding device of this disclosure, the proximal end of the device refers to the end nearest to the handle and the distal end refers to the opposite end, the end nearest to the vent port.

“Fluiid” is used in its broadest sense, to refer to any fluid, including both liquids and gases as well as solutions, compounds, suspensions, etc., which generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially equivalent” is recited with respect to a feature, it is understood that in further embodiments, the feature can have a precisely equivalent configuration.

The terms “a” and “an” can be described as one, but not limited to one. For example, although the disclosure may recite a housing having “a connector,” the disclosure also contemplates that the housing can have two or more connectors.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A catheter infusion system, comprising:
a first balloon catheter device, comprising:
a first catheter comprising:
a distal end portion comprising a vent port;
a first expandable member disposed proximally to the vent port and in fluid communication with the vent port;
a first connector coupled to the catheter;
a handle coupled to the connector, wherein the handle comprises a slide member moveable between a distal position and a proximal position; and
a wire coupled to the slide member and slidingly disposed within the first catheter, wherein a distal portion of the wire is configured to seal the vent port when the slide member is in the distal position and to open the vent port when the slide member is in the proximal position;
a second balloon catheter device, comprising:
a second connector;
a second catheter coupled to the second connector, comprising:
an outer tube;
an inner tube disposed coaxially within the outer tube; and
a second expandable member coupled to the outer tube and disposed adjacent a distal end of the second catheter,
wherein the first catheter is coaxially disposable within the inner tube; and
a backflow valve member coupled to the second connector.

2. The catheter infusion system of claim 1, wherein the first expandable member is inflatable with a fluid when the slide member is in the distal position.

3. The catheter infusion system of claim 1, wherein the expandable member is deflatable when the slide member is in the proximal position.

4. The catheter infusion system of claim 1,
wherein the vent port comprises a lumen comprising a first portion and a second diameter portion, wherein a diameter of the second portion is smaller than a diameter of the first portion,
wherein the distal portion of the wire is configured to seal the vent port when disposed within the second portion, and
wherein the distal portion of the wire is configured to open the vent port when disposed within the first portion.

5. The catheter infusion system of claim 4, wherein the lumen comprises an open distal end configured to vent fluid from the expandable member into an exterior environment when the distal portion of the wire is disposed within the first portion.

6. The catheter infusion system of claim 1,
wherein the second connector comprises a second inflation port,
wherein the outer tube and the inner tube define an annular space between the outer tube and the inner tube, and
wherein the annular space is in fluid communication with the second inflation port and the second expandable member.

7. The catheter infusion system of claim 1,
wherein the backflow valve member comprises an infusion port, and
wherein the inner tube comprises a lumen in fluid communication with the infusion port.

8. The catheter infusion system of claim 1,
wherein the second balloon catheter device is slidingly displaceable over the first catheter from a distal end of the first catheter toward a proximal end of the first catheter.

9. The catheter infusion system of claim 1,
wherein the handle is selectively couplable to the first catheter, and
wherein the second balloon catheter device is slidingly displaceable over the first catheter from a proximal end of the first catheter toward a distal end of the first catheter.

10. A catheter treatment system, comprising:
a catheter occlusion device, comprising:
a first tube comprising:
a fluid evacuation port; and
a vessel occlusion member disposed proximally to and in fluid communication with the fluid evacuation port;
a first connector coupled to the elongate tube;
an actuator coupleable to the connector, wherein the actuator comprises a slide member moveable between a first position and a second position; and
a sealing member coupled to the slide member and slidingly disposed within the first tube, wherein a portion of the sealing member prevents fluid from flowing through the fluid evacuation port when the slide member is in the first position and allows fluid to flow through the fluid evacuation port when the slide member is in the second position; and
a catheter infusion device coaxially disposed over the first tube, comprising:
a second connector;
a second tube coupled to the second connector;
a third tube disposed coaxially within the second tube; and
a second vessel occlusion member coupled to the second tube and disposed adjacent a distal end of the second tube,
wherein the first tube is coaxially disposable within the third tube; and
a backflow prevention member coupled to the second connector.

11. The catheter treatment system of claim 10,
wherein the first vessel occlusion member is inflatable when the slide member is in the first position, and
wherein the first vessel occlusion member is deflatable when the slide member is in the second position.

12. The catheter treatment system of claim 10,
wherein a distal end of the sealing member is disposed within a second portion of a bore of the fluid evacuation port when the slide member is in the first position to seal the fluid evacuation port to prevent an inflation fluid from flowing through the fluid evacuation port, and
wherein the distal end of the sealing member is disposed within a first portion of the bore when the slide member is in the second position to open the evacuation port and allow the inflation fluid to flow from the first occlusion member and distally through and from the evacuation port.

13. The catheter treatment system of claim 10, further comprising an annular space disposed between the second tube and the third tube, wherein the annular space is in fluid communication with the second occlusion member and a second inflation port of the second connector.

14. The catheter treatment system of claim 10, wherein the backflow prevention member comprises a valve configured to engage with the first tube when the backflow prevention member is in a closed state.

15. A method of intravascular treatment, comprising:
disposing a catheter occlusion device coaxially over a catheter infusion device;
disposing an actuator of the catheter occlusion device in a first position, wherein a seal member seals a bore of an occlusion member evacuation port of the catheter occlusion device;
inflating a first expandable occlusion member of the catheter occlusion device with a fluid to occlude a vessel;
inflating a second expandable occlusion member of the catheter infusion device with a fluid to occlude the vessel;
moving the actuator from the first position to a second position, wherein the seal member is displaced to open the bore; and
deflating the first expandable occlusion member, wherein the fluid flows from the first expandable occlusion member into the bore and out a distal end of the occlusion member evacuation port.

16. The method of claim 15, further comprising engaging a catheter of the catheter occlusion device with a backflow prevention member.

17. The method of claim 16, further comprising infusing a treatment agent into the vessel through the backflow prevention member and the catheter infusion device.

18. The method of claim 15, wherein deflating the expandable occlusion member comprises a deflation time of between one second and five seconds.

19. The method of claim 15, wherein inflating the first expandable occlusion member or the second expandable occlusion member anchors the catheter infusion device in a portion of the vessel.

20. The method of claim 15, wherein one or more branch vessels intersect with the vessel between the first expandable occlusion member and the second expandable occlusion member.
